(19) United States
(12) Patent Application Publication
(10) Pub. No.: US 2009/0149804 A1
(43) Pub. Date: Jun. 11, 2009

(54) Mother's Milk Container Closure and Attachment Assembly

(75) Inventors: Joel Delman, Chicago, IL (US); Erin Napolitano, Los Angeles, CA (US); Donna Piacenza, Woodside, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: MEDELA HOLDING AG, Baar (CH)

(21) Appl. No.: 12/140,377

(22) Filed: Jun. 17, 2008

Related U.S. Application Data
(63) Continuation of application No. 10/963,437, filed on Oct. 12, 2004, now abandoned.

(51) Int. Cl. A61M 1/06; A61J 9/08; A61J 9/00

(52) U.S. Cl. 604/74; 215/11.6; 215/11.1

(57) ABSTRACT

An integral container closure securely seals the contents, such as breastmilk, within a container as well as provides for the rotary attachment of the container to other structures such as a breastpump.

### FIELD OF THE INVENTION

The present invention generally relates to closure assemblies for containers. In particular, the present invention relates to a closure and attachment assembly of a container for mother's milk including a cap and threaded arrangement.

### BACKGROUND OF THE INVENTION

Current closure assemblies for containers typically only serve one function, which is to seal the contents within the container, some of which are not tight or secure to prevent contamination, degradation or leakage of the contents.

Contamination is especially hazardous for perishable liquids, such as breastmilk. Contamination of breastmilk can present health hazards to an infant. The merits of breast feeding are well documented in the scientific literature. A number of advantages have been noted which include nutritional, immunological, psychological and other general health advantages, so long as the milk is not contaminated. To prevent contamination, breastmilk should be stored only in clean containers, and immediately sealed and stored if the breastmilk is to be fed to an infant subsequent to expressment. Breastmilk can be stored, such as freezing or refrigerated, to be used at a later time so long as the breastmilk is stored in a secure, tight container.

A nursing mother may use multiple containers for breastmilk increasing the risk contamination. A nursing mother may use a container associated with a breastpump, a container to transfer the expressed milk for storage or feeding, and may even use an additional container to measure the amount of breastmilk before feeding, each container providing different closure and attachment assemblies. This process is inefficient and may necessitate additional clean up along with the risk of contamination. Given the health implications for an infant, the cleanliness and sterilization are of significant concern.

There is a need for a closure assembly for a container such that perishable liquids can be efficiently and effectively stored, such as freezing or refrigeration, without contamination. Further, there is a need for a closure assembly that can be formed with or otherwise affixed to the container mouth in a non-screw-on fashion for easy closure, yet does not impede the use of the container in attaching to other structures such as a breastpump for receiving milk, and then a nipple for feeding.

### SUMMARY OF THE INVENTION

A problem associated with closure assemblies of bottles or containers is that they are very often separate from the container. Making the closure as affixed to or formed integral with the container would then seem to eliminate the ability to use some kind of supplementary rotary attachment mechanism for the container mouth, such as a screw-on attachment. The present invention not only provides a closure assembly that is part of the container to securely seal the contents within a container, but also provides the ability of the container to engage with other structures in a rotary attachment. Thus, an object of the present invention is to provide a universal closure in a snap-on cap, but still provide an attachment for the container that is engageable with a breastpump and bottle nipple, for example, in a screw-on fashion. The closure and container attachment yields a container for receiving, storing and feeding breastmilk, eliminating the need for multiple containers and eliminating loose closures.

A further object of the present invention is to provide a snap-on closure that is formed integral with a bottle or container through a hinge, that securely engages to prevent contamination of perishable beverages, such as breastmilk, during storage, for example freezing and refrigeration. The bottle or container mouth has screw-threads for attachment to other devices.

Yet another object of the present invention is to provide a closure and attachment assembly for a container that includes a cap with a frangible or detachable flexible hinge. Detaching the cap after use ensures the same container is not used for the repeat receiving, storing and feeding of breastmilk, which eliminates the risk of contamination. This further eliminates the need for sterilization. Removing the cap also eliminates it as an impediment during feeding.

The present invention will be further appreciated, and its attributes and advantages further understood, with reference to the detailed description below of some presently contemplated embodiments, taken in conjunction with the accompanying drawings, in which:

### BRIEF DESCRIPTION OF THE DRAWINGS

FIG. 1 is a perspective view of a closure and attachment assembly in the form of a container and cap made according to the present invention;

FIG. 2 is a side view of the container of the closure and attachment assembly of FIG. 1 with the cap removed;

FIG. 3 is a sectional view of the closure and attachment assembly of FIG. 1;

FIG. 4 is an assembly view of the closure and attachment assembly as it would be engaged to a breastpump; and

FIG. 5 is a perspective view of the container engaged to a feeding nipple.

### DESCRIPTION OF THE PREFERRED EMBODIMENT

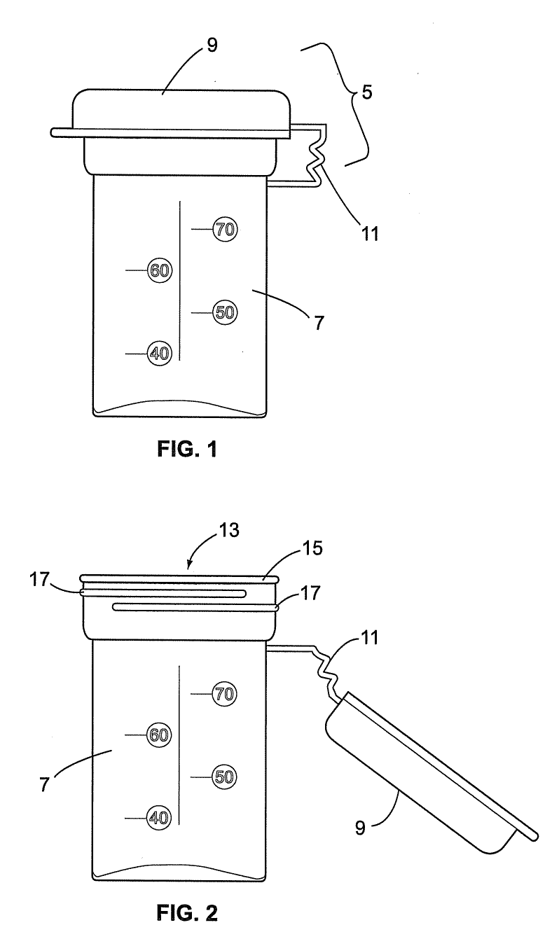

Referring to the drawings and, in particular FIG. 1, there is shown a multifunctional closure and attachment assembly 5. Closure and attachment assembly 5 has a container 7 with a sealing cap 9 coupled thereto via a flexible hinge 11. The closure and attachment assembly 5 is made from plastic, but any rigid or semi-rigid material is contemplated.

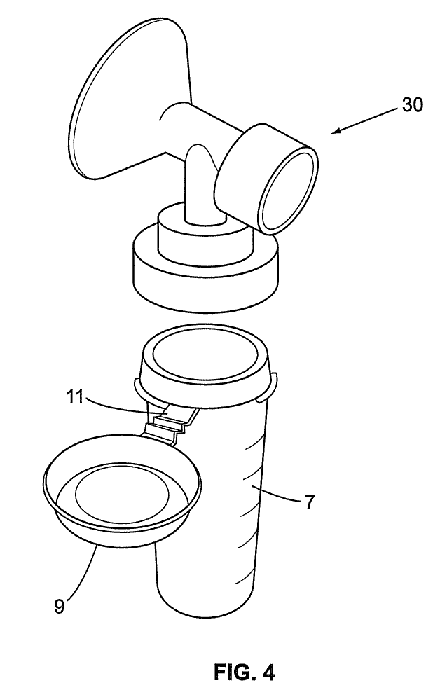

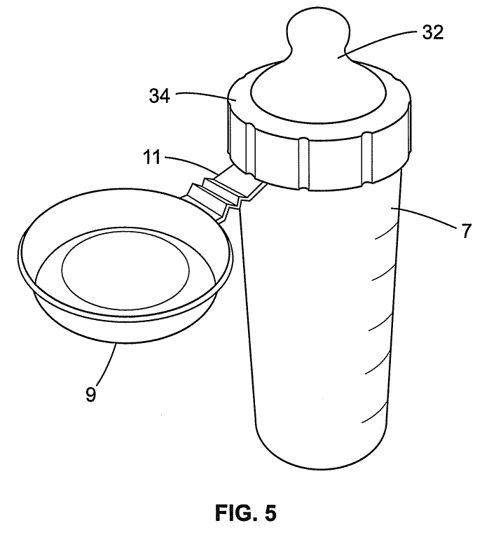

The closure and attachment assembly 5 allows for engagement to a breastpump, 30 for instance (FIG. 4), to express milk from a nursing mother's breast which flows into the container 7. The breastpump is disengaged and the cap 9 seals the container 7 for storage. After storage and upon use, the cap 9 can be left in place, or can be removed from the container 7 by breaking the hinge 11. Detaching the cap after use ensures the same container is not used for the repeat storage of breastmilk, if that is desired. Further, detaching the cap may eliminate it as an impediment during feeding. This further eliminates the need for re-sterilization. The closure and attachment assembly 5 can also engage to a bottle nipple 32 (FIG. 5), as through a collar 34 such that the breastmilk stored in the container 7 can be fed to an infant.

Container 7 has an upper opening 13 that has a shoulder or lip 15, and has a circumferential threaded outer arrangement 17, as shown in FIG. 2. It is noted that container 7 can be any suitable structure sufficient to receive and retain breastmilk.

The opening 13 and threaded arrangement 17 is configured to connect to and cooperate with a breastpump (FIG. 4). The opening 13 and threaded arrangement 17 not only cooperates with the breastpump, but integrally connects the same to facilitate the effective and efficient transfer of breast milk from a nursing mother's breast to the container 7. The opening 13 and threaded arrangement 17 is also configured to connect to and cooperate with a feeding nipple and collar arrangement (FIG. 5). Opening 13 and threaded arrangement 17 can be of any size or shape necessary to effectively connect with a breastpump and feeding nipple in a conventional screw-on fashion.

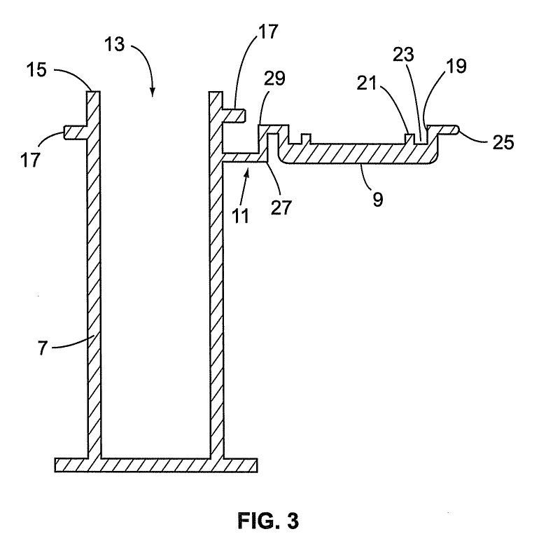

In reference to FIG. 3, cap 9 has a sidewall 19 and annular ring 21 that creates a channel 23. Annular ring 21 cooperates with the lip or shoulder 15 of opening 13 of container 7 to engage and seal the opening 13 with the cap 9 in a snap-fit. Sealing cap 9 can have a different configuration and be formed of any suitable material sufficient to securely connect the cap 9 with the container 7 to seal fluid therein.

Cap 9 has a tab 25 that projects outwardly from sidewall 19 to facilitate a user's ability to connect and disconnect the cap 9 with the container 7. Cap 9 remains coupled to the container 7 via the flexible hinge 11. The flexible hinge 11 integrally and flexibly connects the cap 9 to the container 7. Hinge 11 has two pivot points 27, 29. The cap 9 can easily be detached from the container 7 by breaking either pivot point 27, 29 (see FIG. 3), if so desired.

To seal contents in the container 7, the cap 9 is placed over the opening 13 by pushing down toward the container 7 on tab 25, although pushing down on any location on the cap 9 itself is contemplated to seal the container 7. The opening 13 of the container 7 is sealed when channel 23 of cap 9 engages with shoulder 15.

The hinge is located and designed so that the cap can be swung far enough away from the opening 13 to permit the easy use of the screw-threaded attachment for the container that is also provided.

While particular reference is made to a breastpump environment, there are many other applications to which the present invention may apply. While the apparatus and method herein disclosed form a preferred embodiment of this invention, this invention is not so limited, and changes can be made therein without departing from the scope of this invention which is defined in the appended claims. Those of skill in this art will recognize potential features/designs which can be used in other embodiments, and even more variations, applications and modifications that will still fall within the spirit and scope of the invention, all as intended to come within the ambit and reach of the following claims.

1. A breastpump and container assembly, wherein said container has an opening with an attachment device formed therewith for a rotary attachment of said container to the breastpump, comprising:
a cap engageable with said container to seal said opening in a snap-fit; and
a coupling for said cap to said container.

2. The breastpump and container assembly of claim 1 wherein said container attachment device are screw threads.

3. The breastpump and container assembly of claim 1 wherein said cap is integrally coupled to said container through a flexible hinge.

4. A breastpump and container assembly for collecting and dispensing mother's milk comprising:
a container having an opening with a shoulder and a threaded connector surrounding said container adjacent said opening for attachment to the threaded collar for a breastpump to collect milk, and also for attachment to a feeding nipple having a threaded collar for dispensing milk;
a cap having a sidewall and annular ring forming a channel to receive said shoulder in a snap fit; and
a flexible coupling for said cap to said container.

5-6. (canceled)

7. The breastpump and container assembly of claim 4 wherein said coupling is frangible.
